SEQUENCE LISTING (10 sequences)

SEQ ID NO: 1 - protein, 5 aa, Homo sapiens
  MISC_FEATURE: Antibodies A-E VH CDR1
SYTMH

SEQ ID NO: 2 - protein, 17 aa, Homo sapiens
  MISC_FEATURE: Antibodies A-E VH CDR2
  MISC_FEATURE (8)..(8): Xaa is Arg or Cys
FISYDGNXKYYADSVKG

SEQ ID NO: 3 - protein, 9 aa, Homo sapiens
  MISC_FEATURE: Antibodies A-E VH CDR3
TGWLGPFDY

SEQ ID NO: 4 - protein, 12 aa, Homo sapiens
  MISC_FEATURE: Antibodies A-E VL CDR1
  MISC_FEATURE (5)..(5): Xaa is Ser or Cys
  MISC_FEATURE (8)..(8): Xaa is Ser or Cys
RASQXVGXSYLA

SEQ ID NO: 5 - protein, 7 aa, Homo sapiens
  MISC_FEATURE: Antibodies A-E VL CDR2
  MISC_FEATURE (3)..(3): Xaa is Phe or Cys
  MISC_FEATURE (7)..(7): Xaa is Thr or Cys
GAXSRAX

SEQ ID NO: 6 - protein, 9 aa, Homo sapiens
  MISC_FEATURE: Antibodies A-E VH CDR3
QQYGSSPWT

SEQ ID NO: 7 - protein, 448 aa, Homo sapiens
  MISC_FEATURE: Reference Antibody (heavy chain)
  MISC_FEATURE (1)..(118): Variable region
  MISC_FEATURE (31)..(35): CDR1
  MISC_FEATURE (50)..(66): CDR2
  MISC_FEATURE (99)..(107): CDR3
  MISC_FEATURE (119)..(448): Constant region
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 215 aa, Homo sapiens
  MISC_FEATURE: Reference Antibody (kappa light chain)
  MISC_FEATURE (1)..(108): Variable region
  MISC_FEATURE (24)..(35): CDR1
  MISC_FEATURE (51)..(57): CDR2
  MISC_FEATURE (90)..(98): CDR3
  MISC_FEATURE (109)..(215): Constant region
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 448 aa, Homo sapiens
  MISC_FEATURE: Antibodies A1-E1 (heavy chain)
  MISC_FEATURE (1)..(118): Variable region
  MISC_FEATURE (31)..(35): CDR1
  MISC_FEATURE (50)..(66): CDR2
  MISC_FEATURE (57)..(57): Xaa is Asn or Cys
  MISC_FEATURE (99)..(107): CDR3
  MISC_FEATURE (119)..(448): Constant region
QVQLVESGGGVVQPGRSLRLSCAASGFTFSSYTMHWVRQAPGKGLEWVTFISYDGNXKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Homo sapiens
  MISC_FEATURE: Antibodies A1-E1 (kappa light chain)
  MISC_FEATURE (1)..(108): Variable region
  MISC_FEATURE (24)..(35): CDR1
  MISC_FEATURE (28)..(28): Xaa is Ser or Cys
  MISC_FEATURE (31)..(31): Xaa is Ser or Cys
  MISC_FEATURE (51)..(57): CDR2
  MISC_FEATURE (53)..(53): Xaa is Phe or Cys
  MISC_FEATURE (57)..(57): Xaa is Thr or Cys
  MISC_FEATURE (90)..(98): CDR3
  MISC_FEATURE (109)..(215): Constant region
EIVLTQSPGTLSLSPGERATLSCRASQXVGXSYLAWYQQKPGQAPRLLIYGAXSRAXGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0251206 A1
(43) Pub. Date: Aug. 11, 2022

(54) ANTI-CTLA4 ANTIBODY PRODRUGGABLE (PROBODY) AT A CDR POSITION

(71) Applicant: BRISTOL-MYERS SQUIBB COMPANY, Princeton, NJ (US)

(72) Inventors: Stanley R. KRYSTEK JR., Ringoes, NJ (US); Chetana RAO-NAIK, Walnut Creek, CA (US); Gregory D. VITE, Titusville, NJ (US); Paul E. MORIN, Pennington, NJ (US); Zheng LIN, North Wales, PA (US)

(21) Appl. No.: 17/617,552

(22) PCT Filed: Jun. 8, 2020
(86) PCT No.: PCT/US2020/036574

§ 371 Date: Dec. 8, 2021

Related U.S. Application Data
(60) Provisional application No. 62/859,835, filed on Jun. 11, 2019.

(51) Int. Cl. C07K 16/28; A61P 35/00

(52) U.S. Cl. CPC C07K 16/2818; A61P 35/00; A61K 2039/505; C07K 2317/515; C07K 2317/24

(57) ABSTRACT

An anti-CTLA4 antibody can be prodrugged by replacement of certain complementarity determining region (CDR) amino acids with a cysteine (Cys), which provides a chemical functionality for attachment of a blocking moiety that can be cleaved at the site of intended action, thus releasing the un-prodrugged antibody.

### CROSS-REFERENCE TO RELATED APPLICATIONS

This application claims the benefit under 35 U.S.C. § 119(e) of U.S Provisional Application Ser. No. 62/859,835, filed Jun. 11, 2019; the disclosure of which is incorporated herein by reference.

### SEQUENCE LISTING

Incorporated herein by reference in its entirety is a Sequence Listing named “200428_SEQT_13223WOPCT_YC.txt,” comprising SEQ ID NO:1 through SEQ ID NO:10, which include nucleic acid and/or amino acid sequences disclosed herein. The Sequence Listing has been submitted herewith in ASCII text format via EFS-Web, and thus constitutes both the paper and computer readable form thereof. The Sequence Listing was first created using PatentIn 3.5 on Jan. 1, 2019, and is approximately 16 KB in size.

### BACKGROUND OF THE DISCLOSURE

This application relates to prodruggable antibodies, prodrugs thereof, and methods of making and using such antibodies and their prodrugs.

Therapeutic antibodies can be used to treat a variety of diseases, especially cancer and inflammatory conditions. Examples of therapeutic antibodies that have received marketing approval from regulatory authorities include ipilimumab (YERVOY®), nivolumab (OPDIVO®), trastuzumab (HERCEPTIN®), cetuximab (ERBITUX®), rituximab (RITUXAN®), infliximab (REMICADE®), and adalimumab (HUMIRA®). Generally, a therapeutic antibody—like other antibodies—acts by binding with high specificity for and affinity to its molecular target (the antigen), to initiate the cellular processes related to its therapeutic action.

A prodrug can be used to reduce a therapeutic agent's off-target side effects. A prodrug is a version of a therapeutic agent that is less active, but which can be converted at or near the target tissue or organ into the active therapeutic agent. Commonly, prodrugging is achieved by covalently attaching to the therapeutic agent a moiety that reduces its activity. Removal of the blocking moiety at the target site by a factor or agent found there—low pH, an enzyme, anoxia, etc.—restores the activity of the therapeutic agent. See, for example: Trouet et al. 2004, Stagliano et al. 2013, Rodeck et al. 2010, and Lauermann 2014.

As with the case of other therapeutic agents such as small molecule drugs, it is desirable that the side effects of a therapeutic antibody be prodrugged to reduce or eliminate its action on a tissue or organ other than the one targeted for disease treatment. Classically, an antibody is a Y-shaped dimeric protein, each dimer half consisting of two chains, a heavy and a light chain, as shown in FIG. 1. Commonly, the two dimer halves are identical and are covalently linked to each other by disulfide bonds. The binding interactions of an antibody with its antigen occur through the antibody's complementarity determining regions (CDRs), of which there are three (CDR1, CDR2, and CDR3) on each heavy and light chain, in the variable regions thereof, for a total of six. The heavy and light chain variable regions (labeled V_{H} and V_{L}, respectively in FIG. 1) are located near the amino terminus of each protein chain. Thus, the six CDRs are referred to as V_{H} CDR1, V_{H} CDR2, V_{H} CDR3, V_{L} CDR1, V_{L} CDR2, and V_{L} CDR3. Flanking the CDRs are four framework regions (FR1, FR2, etc.) that provide a scaffold for the CDRs, so that the heavy and light chain variable regions have the N-to-C arrangement FR1-CDR1-FR2-CDR2-FR3-CDR3-FR4.

For prodrugging an antibody, the V_{H} and V_{L} regions are potential sites for attachment of the blocking moiety due to the presence there of the CDRs responsible for antigen interactions. For example, Polu and Lowman 2014 disclose prodrugging an antibody by attaching a masking peptide to the N-terminus of the light chain of an antibody. Other disclosures relating to the prodrugging of antibodies include: Stagliano et al. 2016, Williams et al. 2015, Lowman et al. 2014, Lowman et al. 2015b, and Daugherty et al. 2015. Specific antibodies that have been prodrugged include those against these antigens: EGFR (Desnoyers et al. 2013, Lowman et al. 2015a, Lowman et al. 2017), JAGGED 1/2 (West et al. 2015), interleukin-6 receptor (West et al. 2016a), tissue factor pathway inhibitor (Wang et al. 2016), CD3 (Dennis et al. 2016), PDL1 (West et al. 2016b), CD166 (West et al. 2016c), CD71 (Sagert et al. 2016b), PD1 (Tipton et al. 2017), and ITGA3 (Sagert et al. 2016a).

Krystek et al., U.S. application Ser. No. 16/103,654, filed Aug. 14, 2018, discloses prodrugged antibodies in which a framework amino acid has been replaced by a Cys that serves as an attachment site for a blocking moiety. The linker by which the blocking moiety is attached is cleavable at the site of intended action to release the un-prodrugged antibody.

Some of the documents discussed herein are cited by first author or inventor and year of publication. Their full bibliographic citations are listed in the REFERENCES section towards the end of this specification.

### BRIEF SUMMARY OF THE DISCLOSURE

A cancer treatment that is the subject of intense current interest is immune oncology, in which a cancer patient's own immune system is mobilized to attack the cancer. Although the immune system's cytotoxic T cells can be activated to kill cancer cells, checkpoint inhibitors provide a negative feedback that prevents their activation. One checkpoint inhibitor is CTLA4, which is expressed by activated Tcells. The binding of CTLA4 to its ligand on an antigen-presenting cell initiates the negative feedback signal. The anti-CTLA4 antibody ipilimumab (YERVOY®) has been shown to turn off the inhibitory mechanism by binding to CTLA4, thereby allowing cytotoxic T cell activation and killing of cancer cells. Ipilimumab has been approved for treatment of cancers such as melanoma. As with other therapeutic antibodies, it is desirable that the activity of an anti-CTLA4 antibody be confined to the site of intended action. One method for such confinement would be prodrugging.

The highly variable amino acid sequences in the CDRs accounts for the ability of antibodies to bind to huge variety of antigens, ranging from peptides to carbohydrates to small molecules of non-biologic origin to even metals. Because of the highly specific nature of the binding interactions of their amino acids with the antigen, the CDR amino acids of an antibody are disfavored candidates for replacement by another amino acid for prodrugging purposes, lest the binding interactions be disrupted. Thus, prodrugging efforts have focused on modifications at the amino terminus or in a framework region, as discussed above.

We have discovered that, unexpectedly, replacement of certain CDR amino acids in an anti-CTLA4 antibody with a Cys does not obviate the antibody's ability to bind CTLA4, but yet provides a chemical functionality for attachment of a cleavable blocking moiety to create a prodrugged antibody.

Thus, in one embodiment, there is provided an anti-CTLA4 antibody having
(i) a heavy chain CDR1 comprising the sequence of amino acids of SEQ ID NO:1,
(ii) a heavy chain CDR2 comprising the sequence of amino acids of SEQ ID NO:2,
(iii) a heavy chain CDR3 comprising the sequence of amino acids of SEQ ID NO:3,
(iv) a light chain CDR1 comprising the sequence of amino acids of SEQ ID NO:4,
(v) a light chain CDR2 comprising the sequence of amino acids of SEQ ID NO:5, and
(iv) a light chain CDR3 comprising the sequence of amino acids of SEQ ID NO:6;
   wherein one of Xaa at position 8 of SEQ ID NO:2, at position 5 of SEQ ID NO:4, at position 8 of SEQ ID NO:4, at position 3 of SEQ ID NO:5, or at position 7 of SEQ ID NO:5 is Cys. (The remaining Xaa's being other than Cys.)

In a first embodiment of the aforesaid antibody, Xaa at position 8 of SEQ ID NO:2 is Cys. In a second embodiment of the aforesaid antibody, Xaa at position 5 of SEQ ID NO:4 is Cys. In a third embodiment of the aforesaid antibody, Xaa at position 8 of SEQ ID NO:4 is Cys. In a fourth embodiment of the aforesaid antibody, Xaa at position 3 of SEQ ID NO:5 is Cys. In a fifth embodiment of the aforesaid antibody, Xaa at position 7 of SEQ ID NO:5 is Cys.

In one embodiment, there is provided a prodrugged antibody according to formula (I)

(BM−L)ₘ−Ab (I)

wherein

Ab is an antibody having
(i) a heavy chain CDR1 comprising the sequence of amino acids of SEQ ID NO:1,
(ii) a heavy chain CDR2 comprising the sequence of amino acids of SEQ ID NO:2,
(iii) a heavy chain CDR3 comprising the sequence of amino acids of SEQ ID NO:3,
(iv) a light chain CDR1 comprising the sequence of amino acids of SEQ ID NO:4,
(v) a light chain CDR2 comprising the sequence of amino acids of SEQ ID NO:5, and
(iv) a light chain CDR3 comprising the sequence of amino acids of SEQ ID NO:6; wherein one of Xaa at position 8 of SEQ ID NO:2, at position 5 of SEQ ID NO:4, at position 8 of SEQ ID NO:4, at position 3 of SEQ ID NO:5, or S at position 7 of SEQ ID NO:5 is Cys;

BM is a blocking moiety that inhibits binding of Ab to its antigen;

each L is, independently, a linker moiety bonded to BM and Ab, L comprising a cleavable moiety and being bonded to Ab at a Cys at position 8 of SEQ ID NO:2, at position 5 of SEQ ID NO:4, at position 8 of SEQ ID NO:4, at position 3 of SEQ ID NO:5, or at position 7 of SEQ ID NO:5; and

m is 1 or 2.

### BRIEF DESCRIPTION OF THE DRAWING(S)

FIG. 1 shows the general architecture of an antibody, including the location of the heavy and light chain variable regions (V_{H} and V_{L}), where Cys substitutions disclosed herein are made.

FIG. 2 shows the variable region V_{H} (SEQ ID NO:7, amino acids 1-118) of an anti-CTLA4 antibody, annotated to correlate amino acid numbering in SEQ ID NO:7 and in the Kabat system and to mark desirable location(s) for a Cys substitution in a CDR.

FIG. 3 shows the variable region V_{L} (SEQ ID NO:8, amino acids 1-108) of the light chain of an anti-CTLA4 antibody, annotated to correlate amino acid numbering in SEQ ID NO:8 and in the Kabat system and to mark desirable location(s) for a Cys substitution in a CDR.

FIG. 4, FIG. 5, FIG. 6, FIG. 7, and FIG. 8 show how antibodies disclosed herein can be prodrugged to inhibit binding to CTLA4 and then have their binding ability restored upon removal of the prodrugging group.

FIG. 9, FIG. 10, FIG. 11, FIG. 12, and FIG. 13 show the effect of prodrugging and de-prodrugging of a CTLA4 antibody disclosed herein on IL-2 secretion by PBMC cells.

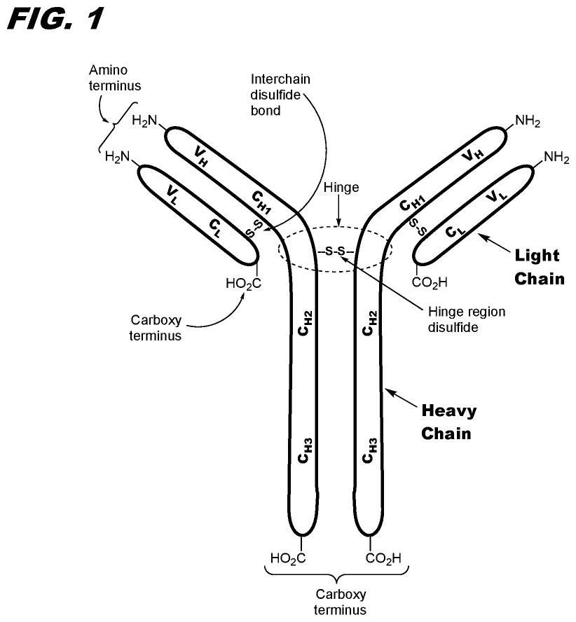

### DETAILED DESCRIPTION OF THE INVENTION

### Definitions

“Antibody” means whole antibodies and any antigen binding fragment (i.e., “antigen-binding portion”) or single chain variants thereof. A whole antibody is a protein comprising at least two heavy (H) chains and two light (L) chains inter-connected by disulfide bonds. Each heavy chain comprises a heavy chain variable region (V_{H}) and a heavy chain constant region comprising three domains, C_{H1}, C_{H2} and C_{H3}. Each light chain comprises a light chain variable region (V_{L} or Vₖ) and a light chain constant region comprising one single domain, C_{L}. The V_{H} and V_{L} regions can be further subdivided into regions of hypervariability, termed complementarity determining regions (CDRs), interspersed with more conserved framework regions (FRs). Each V_{H} and V_{L} comprises three CDRs and four FRs, arranged from amino- to carboxy-terminus in the following order: FR1, CDR1, FR2, CDR2, FR3, CDR3, and FR4. The variable regions contain a binding domain that interacts with an antigen. The constant regions may mediate the binding of the antibody to host tissues or factors, including various cells of the immune system (e.g., effector cells) and the first component (Clq) of the classical complement system. An antibody is said to “specifically bind” to an antigen X if the antibody binds to antigen X with a K_{D} of 5×10⁻⁸ M or less, more preferably 1×10⁻⁸M or less, more preferably 6×10⁻⁹ M or less, more preferably 3×10⁻⁹ M or less, even more preferably 2×10⁻⁹M or less. The antibody can be chimeric, humanized, or, preferably, human. The heavy chain constant region can be engineered to affect glycosylation type or extent, to extend antibody half-life, to enhance or reduce interactions with effector cells or the complement system, or to modulate some other property. The engineering can be accomplished by replacement, addition, or deletion of one or more amino acids or by replacement of a domain with a domain from another immunoglobulin type, or a combination of the foregoing.

“Antigen binding fragment” and “antigen binding portion” of an antibody (or simply “antibody portion” or “antibody fragment”) mean one or more fragments of an antibody that retain the ability to specifically bind to an antigen. It has been shown that the antigen-binding function of an antibody can be performed by fragments of a full-length antibody, such as (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fab' fragment, which is essentially an Fab with part of the hinge region (see, for example, Abbas et al., Cellular and Molecular Immunology, 6th Ed., Saunders Elsevier 2007); (iv) a Fd fragment consisting of the V_{H} and C_{H1} domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Preferred antigen binding fragments are Fab, F(ab′)₂, Fab′, Fv, and Fd fragments. Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} regions pair to form monovalent molecules (known as single chain Fv, or scFv); see, e.g., Bird et al. (1988) Science 242:423-426; and Huston et al. (1988) Proc. Natl. Acad. Sci. USA 85:5879-5883). Such single chain antibodies are also encompassed within the term “antigen-binding portion” of an antibody.

Unless indicated otherwise—for example by reference to the numbering in a SEQ ID NO: listing—references to the numbering of amino acid positions in an antibody heavy or light chain variable region (V_{H} or V_{L}) are according to the Kabat system (Kabat et al., “Sequences of proteins of immunological interest, 5th ed., Pub. No. 91-3242, U.S. Dept. Health & Human Services, NIH, Bethesda, Md., 1991, hereinafter “Kabat”) and references to the numbering of amino acid positions in an antibody heavy or light chain constant region (C_{H1}, C_{H2}, C_{H3}, or C_{L}) are according to the EU index as set forth in Kabat. See Lazar et al., US 2008/0248028 A1, the disclosure of which is incorporated herein by reference, for examples of such usage. Further, the ImMunoGeneTics Information System (IMGT) provides at its website a table entitled “IMGT Scientific Chart: Correspondence between C Numberings” showing the correspondence between its numbering system, EU numbering, and Kabat numbering for the heavy chain constant region. See, e.g., Lazar et al., US 2008/0248028 A1 (2008).

An “isolated antibody” means an antibody that is substantially free of other antibodies having different antigenic specificities (e.g., an isolated antibody that specifically binds antigen X is substantially free of antibodies that specifically bind antigens other than antigen X). An isolated antibody that specifically binds antigen X may, however, have cross-reactivity to other antigens, such as antigen X molecules from other species. In certain embodiments, an isolated antibody specifically binds to human antigen X and does not cross-react with other (non-human) antigen X antigens. Moreover, an isolated antibody may be substantially free of other cellular material and/or chemicals.

“Monoclonal antibody” or “monoclonal antibody composition” means a preparation of antibody molecules of single molecular composition, which displays a single binding specificity and affinity for a particular epitope.

“Human antibody” means an antibody having variable regions in which both the framework and CDR regions (and the constant region, if present) are derived from human germ-line immunoglobulin sequences. Human antibodies may include later modifications, including natural or synthetic modifications. Human antibodies may include amino acid residues not encoded by human germline immunoglobulin sequences (e.g., mutations introduced by random or site-specific mutagenesis in vitro or by somatic mutation in vivo). However, “human anti-body” does not include antibodies in which CDR sequences derived from the germline of another mammalian species, such as a mouse, have been grafted onto human framework sequences.

“Human monoclonal antibody” means an antibody displaying a single binding specificity, which has variable regions in which both the framework and CDR regions are derived from human germline immunoglobulin sequences. In one embodiment, human monoclonal antibodies are produced by a hybridoma that includes a B cell obtained from a transgenic nonhuman animal, e.g., a transgenic mouse, having a genome comprising a human heavy chain transgene and a light chain transgene fused to an immortalized cell.

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the number of carbons in other types, as in C₂₋₄ alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkylene” means a divalent counterpart of an alkyl group, such as CH₂CH₂, CH₂CH₂CH₂, and CH₂CH₂CH₂CH₂.

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (or Z-) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cycloalkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkylene” means a divalent counterpart of a cycloalkyl group.

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.+

“Aryl” means a hydrocarbon moiety having a mono-, bi-, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi-, or tricyclic ring system (preferably 5- to 7-membered monocyclic) wherein each ring has from 3 to 7 carbon atoms and at least one ring is an aromatic ring containing from 1 to 4 heteroatoms independently selected from from N, 0, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzofuranyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocyclo-aliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF3), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O (alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄alkyoxy, O(C₂-C₄ alkylene)OH, and O(C₂-C₄ alkylene)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O (alkyl), —C(═O)O (hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in C₁ and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature), all stereoisomers are included within the scope of the invention, as pure compounds as well as mixtures thereof. Unless otherwise indicated, individual enantiomers, diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has per se activity similar to that of the parent compound. Suitable esters include C₁-C₅ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methylsulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

In the formulae of this specification, a wavy line ( [symbol] ) transverse to a bond or an asterisk (*) at the end of the bond denotes a covalent attachment site. For instance, a statement that R is

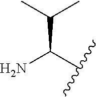 [SMILES: *[C@@H](N)C(C)C]

or that R is

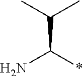 [SMILES: *[C@@H](N)C(C)C]

in the formula

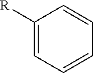

means

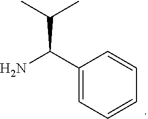

In the formulae of this specification, a bond traversing an aromatic ring between two carbons thereof means that the group attached to the bond may be located at any of the available positions of the aromatic ring. By way of illustration, the formula

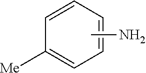

represents

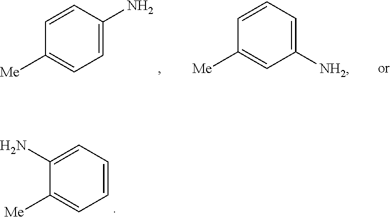 [SMILES: Cc1ccc(N)cc1 ; Cc1cccc(N)c1 ; Cc1ccccc1N]

### EMBODIMENTS

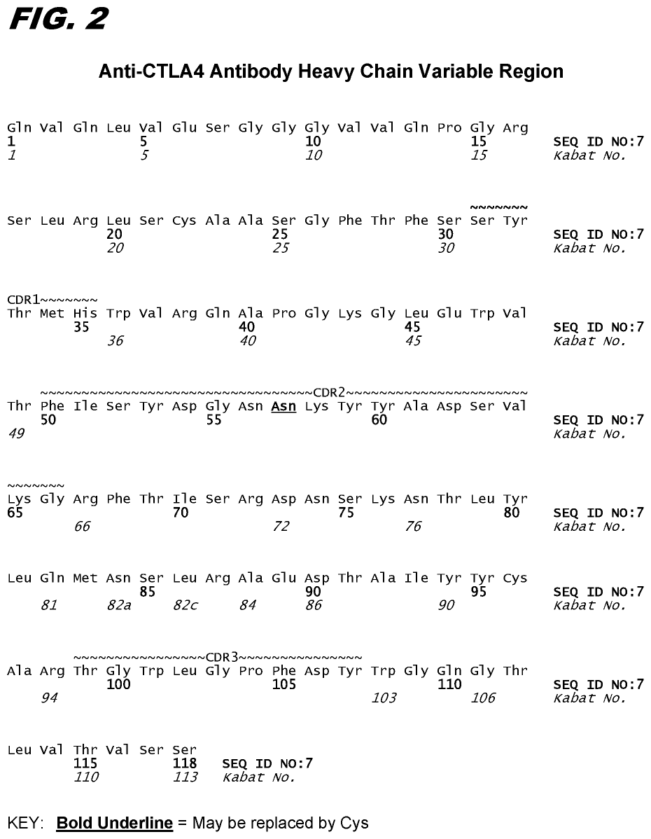

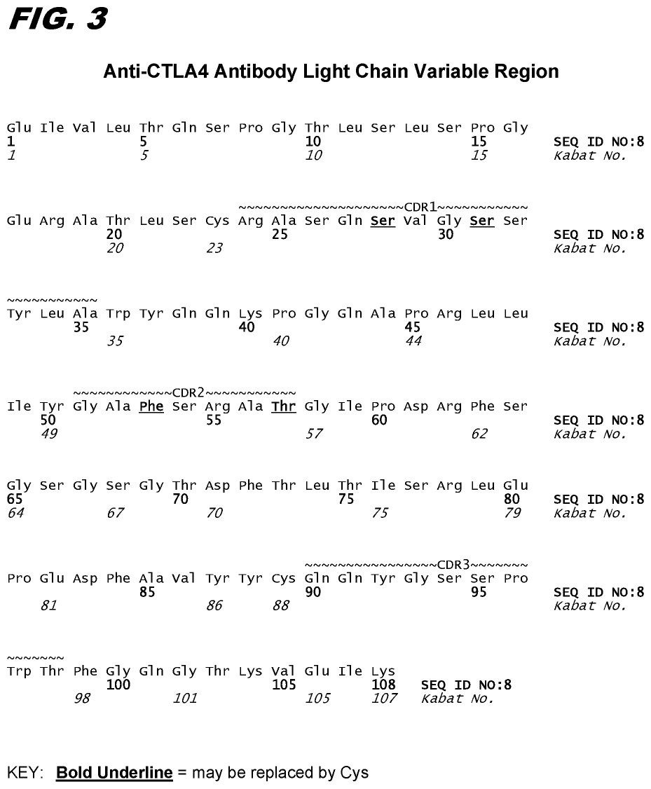

Working with an anti-CTLA4 antibody having the heavy and light chain variable region amino acid sequences shown in FIGS. 2 and 3, respectively, we investigated which CDR amino acids might be amenable substitution with a Cys for attachment of a prodrugging moiety. Effective prodrugging requires: (a) a blocking moiety must be attachable to the substituted-in Cys; (b) the blocking moiety inhibits binding of the antibody to the antigen (in this case, CTLA4); (c) the blocking moiety must be removable (typically by cleavage of the linker attaching it to the Cys); and (d) antigen-binding activity must be restored upon removal of the blocking moiety.

Given the specificity of the interaction of CDR amino acids with the antigen, we found, not surprisingly, that not all CDR amino acids were amenable to such substitution. However, we found, unexpectedly, that several were amenable for prodrugging. These are highlighted in FIGS. 2 and 3.

A first prodruggable anti-CTLA4 antibody, referred to as “Antibody A” has these CDRs:

V_{H} CDR1:
(SEQ ID NO: 1)
SYTMH
V_{H} CDR2:
(SEQ ID NO: 2, Xaa is Cys)
FISYDNCKYYADSVKG
V_{H} CDR3:
(SEQ ID NO: 3)
TGWLGPFDY
V_{K} CDR1:
(SEQ ID NO: 4, each Xaa is Ser)
RASQSVGSSYLA
V_{K} CDR2:
(SEQ ID NO: 5, Xaa at position 5 is Phe and Xaa at
position 7 is Thr)
GAFSRAT
V_{K} CDR3:
(SEQ ID NO: 6)
QQYGSSPWT

A specific example of Antibody A, referred to as Antibody A1, has the full length heavy and light chain amino acid sequences of SEQ ID NO:9 (where Xaa is Cys) and NO:10 (where Xaa at position 28 is Ser, at position 31 is Ser, at position 53 is Phe, and at position 57 is Thr), respectively.

A second prodruggable anti-CTLA4 antibody, referred to as “Antibody B” has these CDRs:

V_{H} CDR1:
(SEQ ID NO: 1)
SYTMH
V_{H} CDR2:
(SEQ ID NO: 2, Xaa is Arg)
FISYDNNKYYADSVKG
V_{H} CDR3:
(SEQ ID NO: 3)
TGWLGPFDY
V_{K} CDR1:
(SEQ ID NO: 4, Xaa at position 5 is Cys, Xaa at
position 8 is Ser)
RASQCVGSSYLA
V_{K} CDR2:
(SEQ ID NO: 5, Xaa at position 3 is Phe and Xaa at
position 7 is Thr)
GAFSRAT
V_{K} CDR3:
(SEQ ID NO: 6)
QQYGSSPWT

A specific example of Antibody B, referred to as “Antibody B1”, has the full length heavy and light chain amino acid sequences of SEQ ID NO:9 (where Xaa is Arg) and NO:10 (where Xaa at position 28 is Cys, at position 31 is Ser, at position 53 is Phe, and at position 57 is Thr), respectively.

A third prodruggable anti-CTLA4 antibody, referred to as “Antibody C”, has these CDRs:

V_{H} CDR1:
(SEQ ID NO: 1)
SYTMH
V_{H} CDR2:
(SEQ ID NO: 2, Xaa is Arg)
FISYDNNKYYADSVKG
V_{H} CDR3:
(SEQ ID NO: 3)
TGWLGPFDY
V_{K} CDR1:
(SEQ ID NO: 4, Xaa at position 5 is Ser and Xaa
at position 8 is Cys)
RASQSVGCSYLA
V_{K} CDR2:
(SEQ ID NO: 5, Xaa at position 3 is Phe and Xaa at
position 7 is Thr)
GAFSRAT
V_{K} CDR3:
(SEQ ID NO: 6)
QQYGSSPWT

A specific example of Antibody C, referred to as “Antibody C1”, has the full length heavy and light chain amino acid sequences of SEQ ID NO:9 (where Xaa is Arg) and NO:10 (where Xaa at position 28 is Ser, at position 31 is Cys, at position 53 is Phe, and at position 57 is Thr), respectively.

A fourth prodruggable anti-CTLA4 antibody, referred to as “Antibody D”, has these CDRs:

V_{H} CDR1:
(SEQ ID NO: 1)
SYTMH
V_{H} CDR2:
(SEQ ID NO: 2, Xaa is Arg)
FISYDNNKYYADSVKG
V_{H} CDR3:
(SEQ ID NO: 3)
TGWLGPFDY
V_{K} CDR1:
(SEQ ID NO: 4, each Xaa is Cys)
RASQSVGSSYLA
V_{K} CDR2:
(SEQ ID NO: 5, Xaa at position 3 is Cys and Xaa at
position 7 is Thr)
GACSRAT
V_{K} CDR3:
(SEQ ID NO: 6)
QQYGSSPWT

A specific example of Antibody D, referred to as “Antibody Dl”, has the full length heavy and light chain amino acid sequences of SEQ ID NO:9 (where Xaa is Arg) and NO:10 (where Xaa at position 28 is Ser, at position 31 is Ser, at position 53 is Cys, and at position 57 is Thr), respectively.

A fifth prodruggable anti-CTLA4 antibody, referred to as “Antibody E”, has these CDRs:

V_{H} CDR1:
(SEQ ID NO: 1)
SYTMH
V_{H} CDR2:
(SEQ ID NO: 2, Xaa is Arg)
FISYDNNKYYADSVKG
V_{H} CDR3:
(SEQ ID NO: 3)
TGWLGPFDY
V_{K} CDR1:
(SEQ ID NO: 4, each Xaa is Ser)
RASQSVGSSYLA
V_{K} CDR2:
(SEQ ID NO: 5, Xaa at position 3 is Phe and Xaa at
position 7 is Cys)
GAFSRAC
V_{K} CDR3:
(SEQ ID NO: 6)
QQYGSSPWT

A specific example of Antibody E, referred to as “Antibody El”, has the full length heavy and light chain amino acid sequences of SEQ ID NO:9 (where Xaa is Arg) and NO:10 (where Xaa at position 28 is Ser, at position 31 is Ser, at position 53 is Phe, and at position 57 is Cys), respectively.

Antibodies A, B, C, D, and E can have either a kappa or lambda light chain. In the antibody species A1, B1, C1, D1, and E1, the light chain is of the kappa type.

Deleting the heavy chain C-terminal lysine is often done when producing antibodies by recombinant expression, to reduce heterogeneity (McDonough et al., U.S. Pat. No. 5,126,250 (1992)). In SEQ ID NO:9, antibodies A1, B1, C1, D1, and E1 are shown with the heavy chain C-terminal lysine 448 present. In another embodiment, antibodies A1, B1, C1, D1, and E1 can have the heavy chain C-terminal lysine deleted, that is, with heavy chain sequences comprising amino acids 1-447 of SEQ ID NO:9.

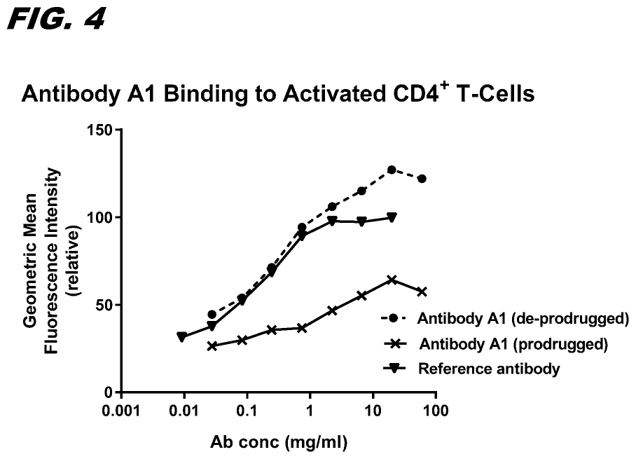

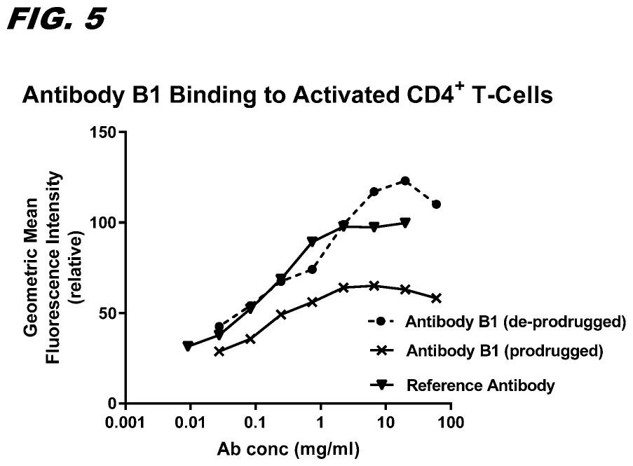

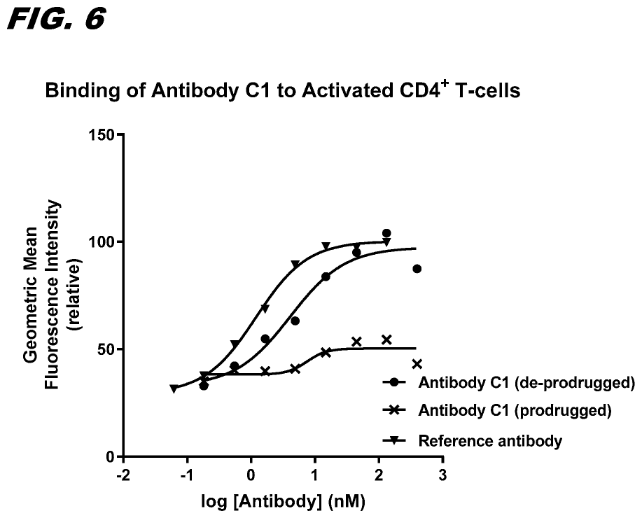

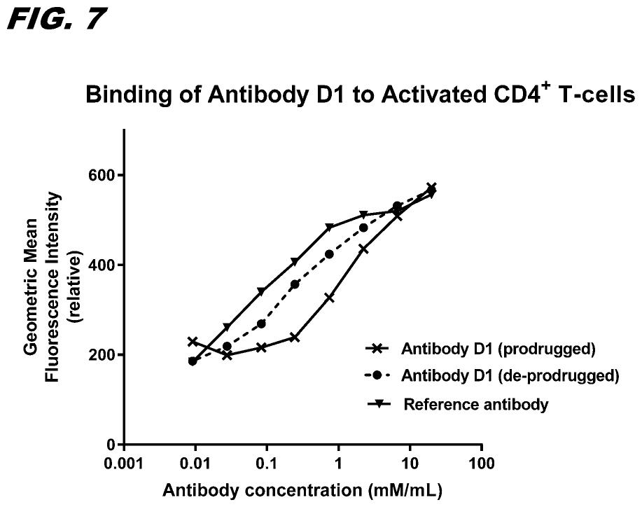

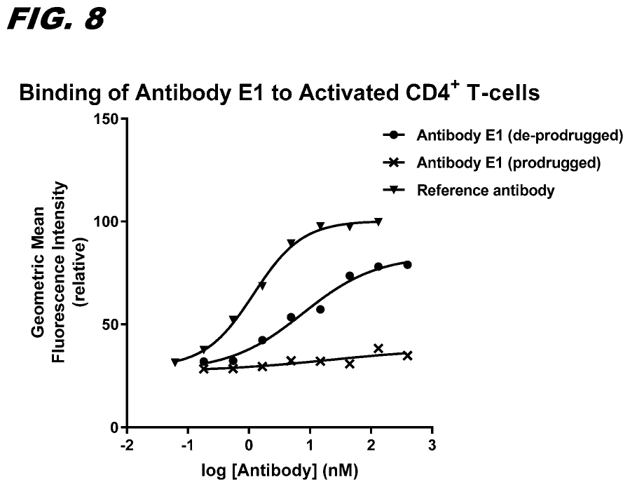

FIGS. 4-8 show how antibodies A1, B1, C1, D1, and E1, upon prodrugging by attachment of a blocking moiety, have reduced ability binding to activated CD⁴⁺ T cells, which express CTLA4, but recover their binding ability to such cells when de-prodrugged by removal of the blocking moiety. In each figure: (a) the —x— graph line shows by prodrugged antibody; (b) the —●— graph line shows the binding by de-prodrugged antibody (i.e., after removal of the prodrugging group; and (c) the —▾— graph line shows binding by a Reference Antibody, which has not been modified by replacement of a CDR amino acid with a Cys.

Not all CDR amino acids were amenable to prodrugging. For instance, we found no prodrugging effect with a light chain S26C substitution.

The aforementioned “Reference Antibody” is an antibody having a heavy chain amino acid sequence according to SEQ ID NO:7 and a light chain amino acid sequence according to SEQ ID NO:8. Its CDRs, as shown below, are identical to those of ipilimumab (YERVOY®, CAS Reg. No. 477202-00-9), an anti-CTLA4 antibody that has obtained marketing approval for treatment of melanoma.

V_{H} CDR1:
(amino acids 31-35, SEQ ID NO: 7)
SYTMH
V_{H} CDR2:
(amino acids 50-66, SEQ ID NO: 7)
FISYDNNKYYADSVKG
V_{H} CDR3:
(amino acids 99-107, SEQ ID NO: 7)
TGWLGPFDY
V_{K} CDR1:
(amino acids 24-35, SEQ ID NO: 8)
RASQSVGSSYLA
V_{K} CDR2:
(amino acids 51-57, SEQ ID NO: 8)
GAFSRAT
V_{K} CDR3:
(amino acids 90-98, SEQ ID NO: 8)
QQYGSSPWT

In one embodiment, the prodruggable anti-CTLA4 antibody is of the IgG1 isotype. Preferably it has the allotype combination of R214 (EU index numbering; amino acid 215 in SEQ ID NO:9), E356 (EU index numbering; amino acid 357 in SEQ ID NO:9), and M358 (EU index numbering; amino acid 359 in SEQ ID NO:9), which combination is common in the Caucasian population.

In one embodiment, the linker comprises a polypeptide that is cleavable by—i.e., is a substrate for—an enzyme (a protease) that is uniquely expressed or overexpressed at the diseased tissue or organ, compared to healthy tissue or organ. Preferably, the enzyme is found in the extracellular environment of the diseased tissue or organ. Examples of such proteases include: aspartate proteases (e.g., renin), fibroblast activation protein (FAP), aspartic cathepsins (e.g., cathepsin D, caspase 1, caspase 2, etc.), cysteine cathepsins (e.g., cathepsin B), cysteine proteases (e.g., legumain), disintegrin/metalloproteinases (ADAMs, e.g., ADAMS, ADAMS), disintegrin/metalloproteinases with thrombospondin motifs (ADAMTS, e.g., ADAMTS1), integral membrane serine proteases (e.g., matriptase 2, MT-SP1/matriptase, TMPRSS2, TMPRSS3, TMPRSS4), kallikrein-related peptidases (KLKs, e.g. KLK4, KLKS), matrix metalloproteases (e.g., MMP-1, MMP-2, MMP-9), and serine proteases (e.g., cathepsin A, coagulation factor proteases such as elastase, plasmin, thrombin, PSA, uPA, Factor VIIa, Factor Xa, and HCV NS3/4). Preferably, the protease is fibroblast activation protein (FAP), urokinase-type plasminogen activator (uPA, urokinase), MT-SP1/matriptase, legumain, or a matrix metalloprotease (especially MMP-1, MMP-2, and MMP-9). Those skilled in the art will appreciate that the choice of the enzyme and the corresponding cleavable peptide will depend on the disease to be treated and the protease(s) expressed by the affected tissue or organ.

Examples of polypeptide substrate-enzyme pairs are provided in TABLE I.

TABLE I
POLYPEPTIDE SUBSTRATES AND CLEAVING ENZYMES

| Cleavable Peptide | SEQ ID NO. | Cleaving Enzyme(s) |
|---|---|---|
| LSGRSDNH | 5 | Urokinase, matriptase, legumain |
| VPLSLYS | 6 | Matrix metalloprotease-9 (MMP-9) |
| PLGLAG | 7 | Matrix metalloprotease-2 (MMP-2) |
| VLVPMAMMAS | 8 | Matrix metalloprotease-1 (MMP-1) |
| XXQAR(A/V)X (where X | 9 | Matriptase (MT-SP1) |
| is any amino acid) | | |
| AGPR | 10 | Matriptase (MT-SP1) |
| AANL | 11 | Legumain |
| PTNL | 12 | Legumain |
| TSGRSANP | 13 | Various |
| DEXXXC(A/S) where X | 14 | HCV NS3/4 |
| is any amino acid) | | |
| DLXXXT(A/S) (where X | 15 | HCV NS3/4 |
| is any amino acid. | | |
| LSGX (where X is R or K) | 16 | Matriptase |

Preferably, the cleavable peptide is LSGRSDNH (SEQ ID NO:5), LSGX (SEQ ID NO:16) or LSGK (SEQ ID NO:16).

Disclosures of suitable proteases and/or their substrates include: Desnoyers et al. 2013; Stagliano et al. 2013; Stagliano et al. 2014; Waldmann et al. 2013; Lauermann et al. 2014; Lowman et al. 2014; Daugherty et al. 2015; Lowman et al. 2015a; Lowman et al. 2015b; Moore et al. 2015; West et al. 2016a; Wang et al. 2016; Moore et al. 2016; Moore et al. 2017; and Dennis et al. 2016; the disclosures of which are incorporated herein by reference.

Blocking moieties BM that can be used to interfere with or block activity of a prodrugged antibody with its antigen include: polyethylene glycol (PEG), an albumin binding polypeptide, adnectin, a peptide, and a soluble globular protein such as albumin or fibrinogen.

In one embodiment, BM is PEG having a molecular weight of at least about 2 kDa, with 2 kDa corresponding to PEG with about 45 —(CH₂CH₂O)— repeating units, and preferably PEG with a molecular weight of at least about 5 kDa, with 5 kDa corresponding to PEG with about 115 —(CH₂CH₂O)— repeating units.

Amine-terminated PEG with 48 —(CH₂CH₂O)— repeating units (CAS Reg. No. 32130-27-1) is available from Quanta Biodesign Ltd. Amine-terminated PEG with a nominal molecular weight of 5 kDa is available from NOF America Corp. (CAS Reg. No. 116164-53-5). Using MALDI-TOF-MS analysis, we determined that it has a molecular weight distribution of between about 4.4 kDa and about 6.6 kDa, corresponding to between about 100 and about 155 —(CH₂CH₂O)— repeating units. Amine-terminated PEG with a nominal molecular weight of 10 kDa is available from NOF America Corp. (CAS Reg. No. also 116164-53-5. Using MALDI-TOF-MS analysis, we determined that it has a molecular weight distribution of between about 9.0 kDa and about 12.0 kDa, corresponding to between about 205 and about 275 —(CH₂CH₂O)— repeating units. The terminal amine group provides a chemical functionality for attachment to the linker moiety.

Disclosures of these and other blocking moieties BM include: Tomasi et al. 1988; Trouet et al. 2004; Waldmann et al. 2014; Lauermann et al. 2014; Lowman et al.2014; Daugherty et al. 2015; Lowman et al. 2015a; West et al. 2016a; and Wang et al. 2016; the disclosures of which are incorporated herein by reference.

An antibody having a Cys as described hereinabove can be conjugated to a blocking moiety-linker moiety compound having a maleimide terminal group by Michael addition of the Cys sulfhydryl (SH), as shown below. The procedures for such conjugation are well known in the art; see, for example, Shepard et al., WO 2017/112624 A1 (2017).

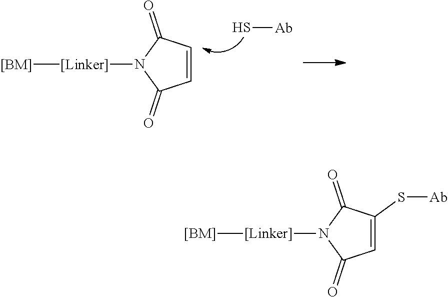

Examples of maleimide terminated blocking moiety-linker compounds that can be so used to prodrug an antibody include ones according to formulae (Ia)-(Ih), each of which comprises a peptide cleavable by the enzyme matriptase. These blocking moiety-linker compounds can be made as disclosed in US Application of Krystek et al., US 2019/0055321 A1, the disclosure of which is incorporated herein by reference.

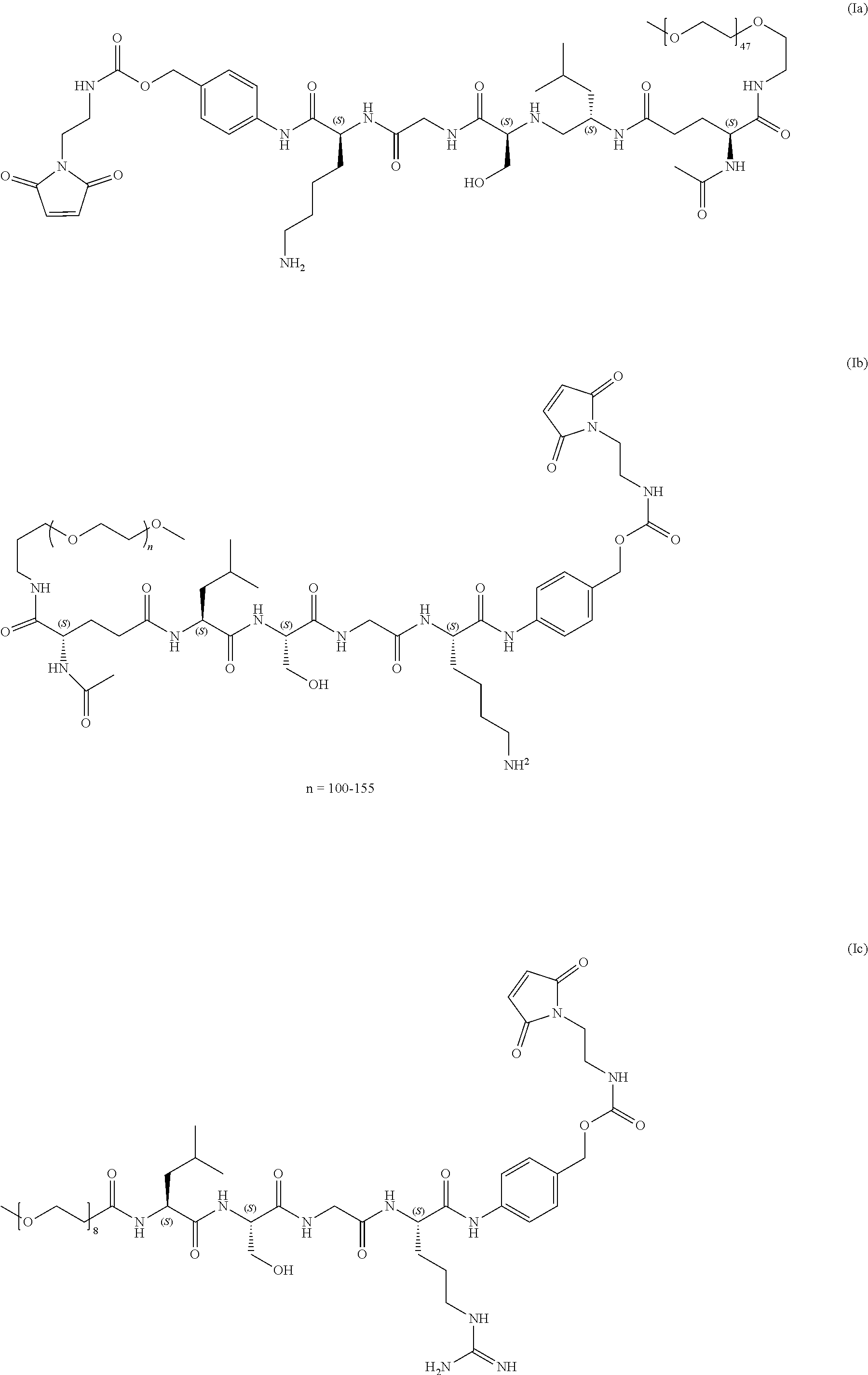

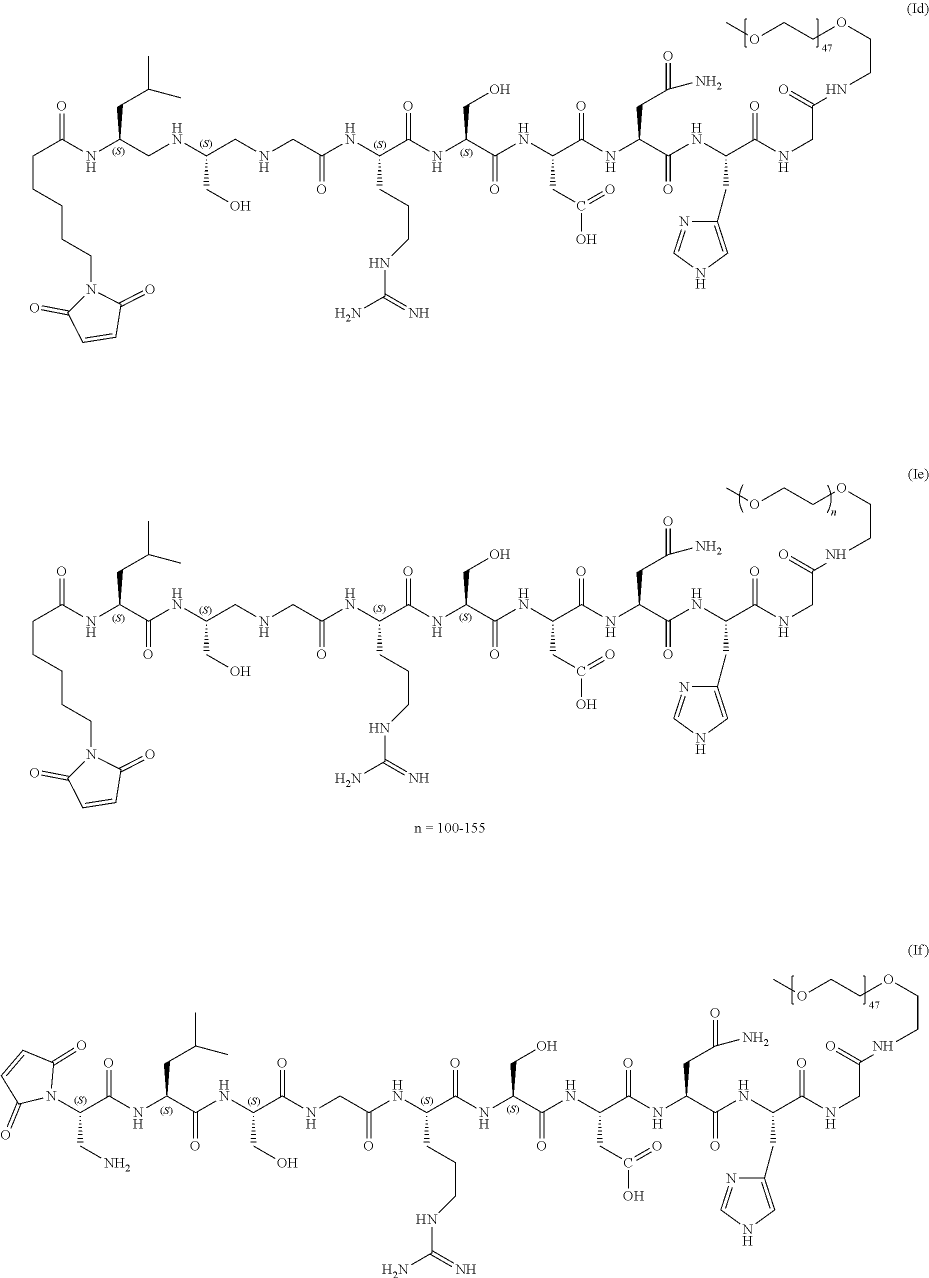

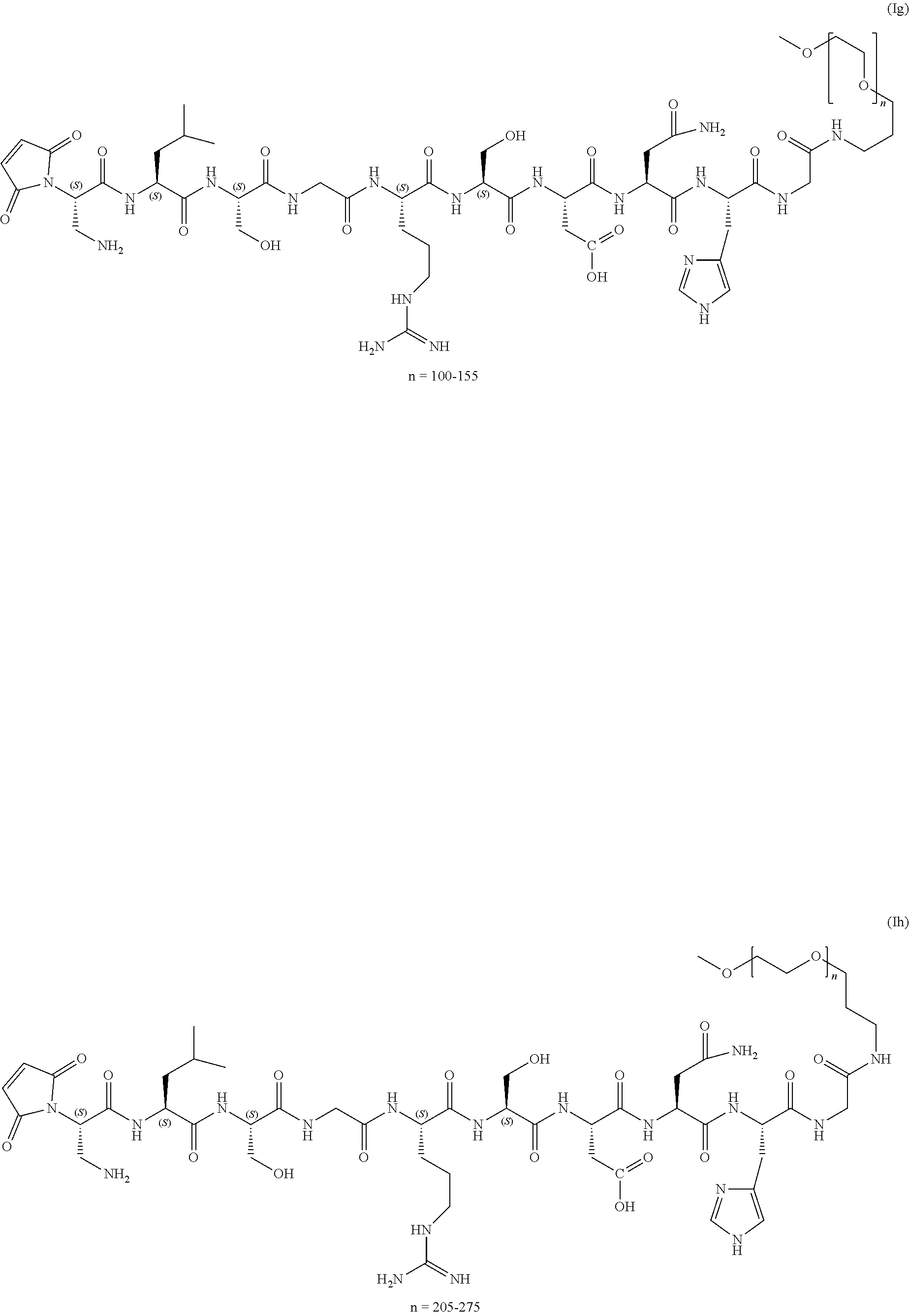

Conjugation of an antibody with the foregoing blocking moiety-linker compounds (Ia)-(Ih) provides prodrugged antibodies according to the formulae (IIa)-(IIh), respectively, where Ab is an antibody as defined above and m is 1 or 2.

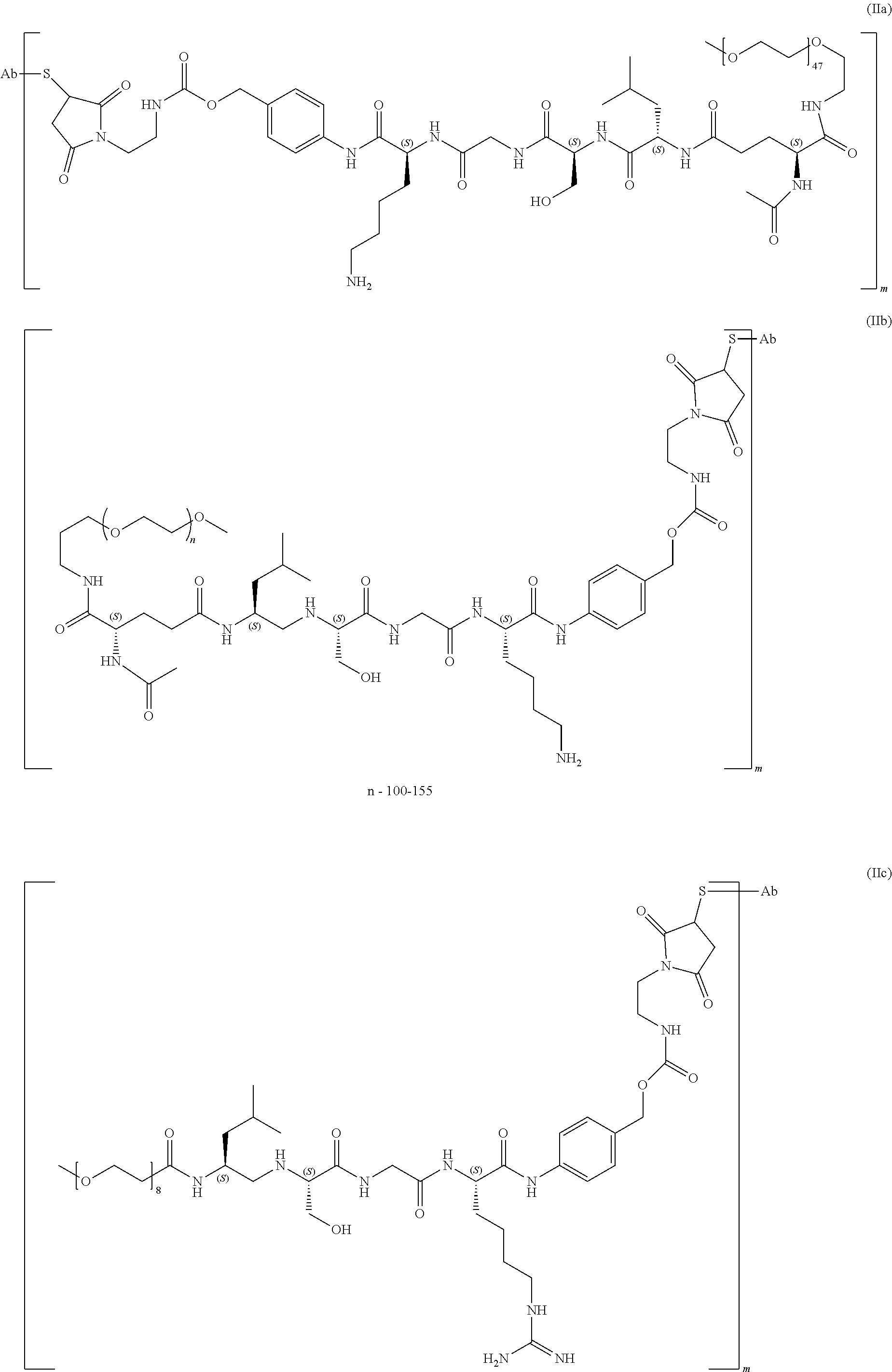

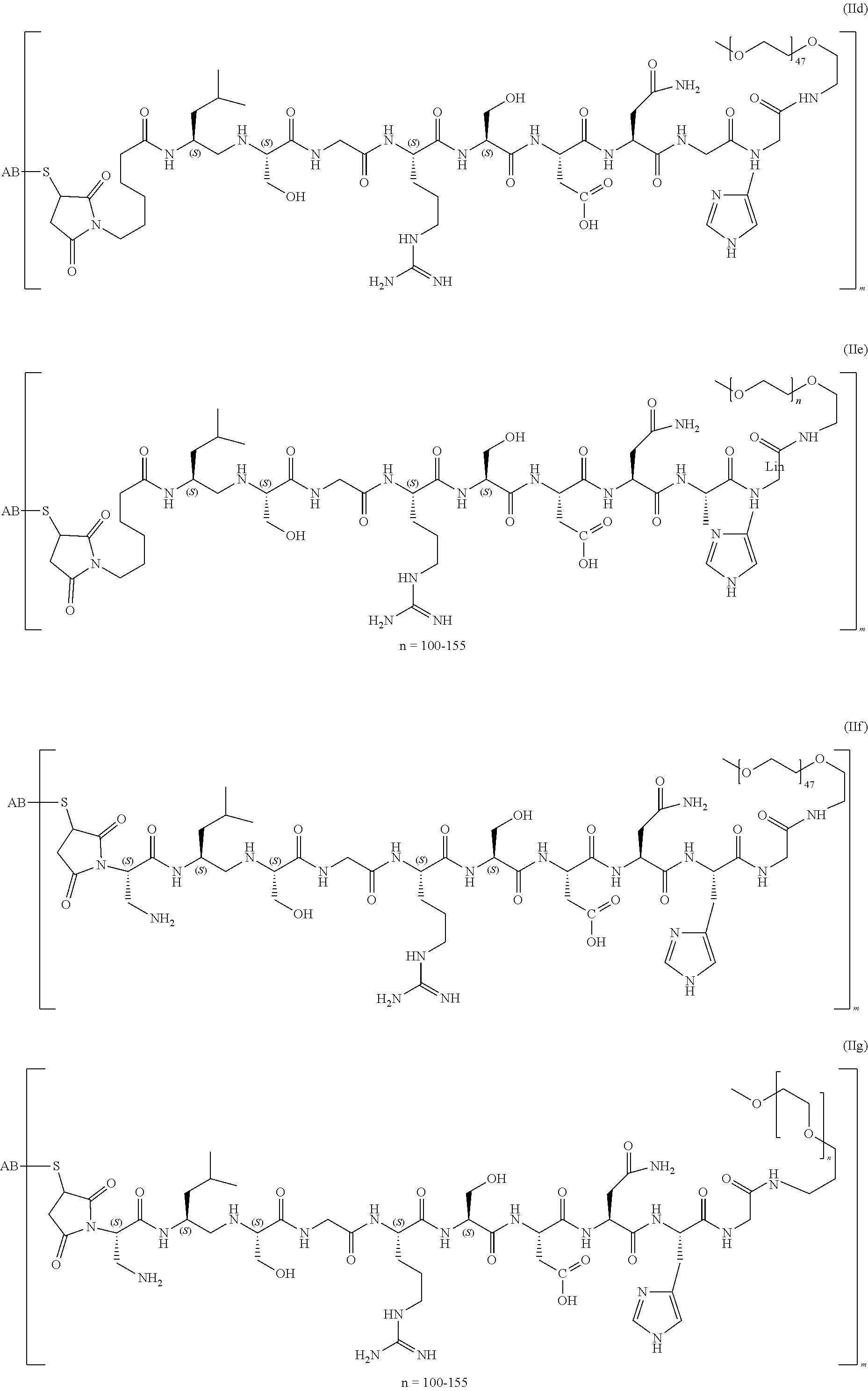

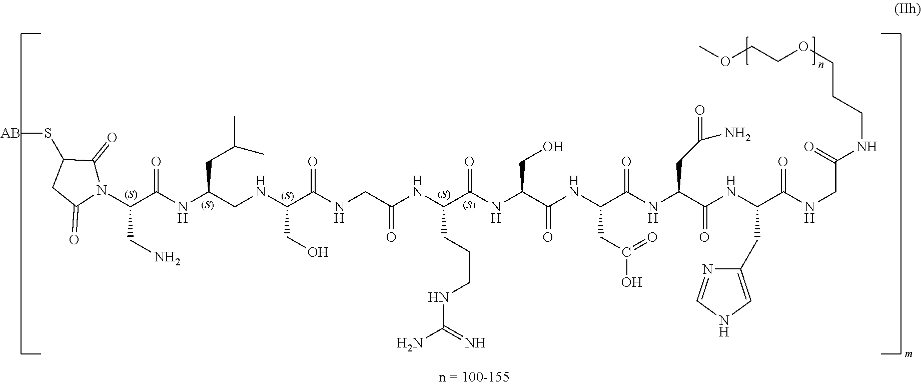

Those skilled in the art will appreciate that, over time, the initially formed succinimide structure resulting from thiol addition to the maleimide group may hydrolytically ring-open to a seco form, and that the succinimide and seco forms are functionally equivalent.

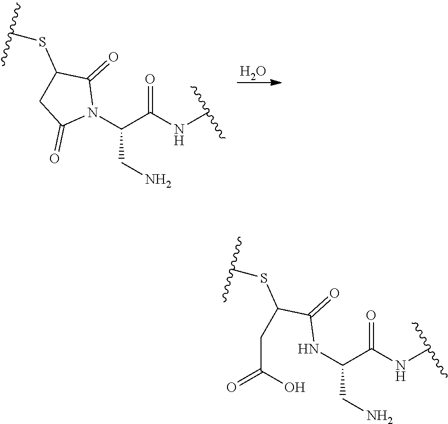 [SMILES: *NC(=O)[C@H](CN)N1C(=O)CC(S*)C1=O ; O ; *NC(=O)[C@H](CN)NC(=O)C(CC(=O)O)S*]

As can be discerned from some of the structures above, the enzymatically cleavable peptide can be used in combination with a self-immolating group. Briefly, the function of a self-immolating group is to provide separation between the peptide and other portions of the antibody, the linker, or the blocking moiety, lest any of them interfere with the action of the cleaving enzyme. After cleavage occurs, the self-immolating group undergoes a self-elimination reaction. Uses and structures of self-immolating groups are described in Zhang et al., U.S. Pat. No. 9,527,871 B2 (2016), the disclosure of which is incorporated herein by reference.

A preferred self-immolating group is ap-aminobenzyl oxycarbonyl (PABC) group, whose structure is shown below.

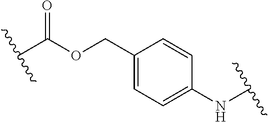 [SMILES: *Nc1ccc(COC(*)=O)cc1]

Its mode of action is illustrated in the reaction sequence below:

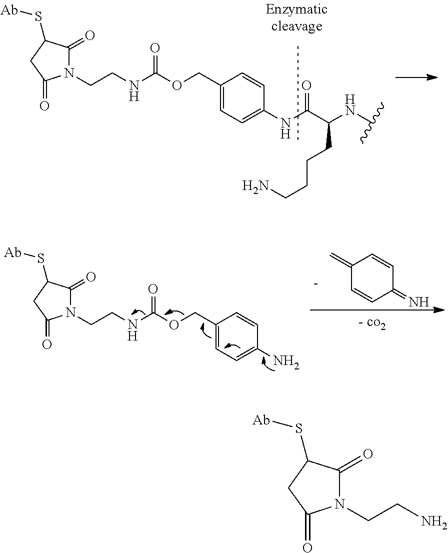

As can be seen from the above reaction sequence, cleavage of the peptide does not restore the antibody Ab to its original structure in the sense that the Cys bonding to the blocking moiety-linker is left with a succinimide residue (or its ring-opened derivative) attached thereto.

This applies regardless of whether a self-immolating group was used or not. We have discovered that, unexpectedly, the succinimide residue does not prevent the restoration of the antibody's antigen binding activity.

### EXAMPLES

The practice of this invention can be further understood by reference to the following examples, which are provided by way of illustration and not of limitation.

A table after the Examples lists acronyms and abbreviations used herein and their meanings.

### Example 1—Conjugation

Antibodies having selected Cys substitutions were transiently expressed in Expi-CHO cells and purified using standard protocols with protein A chromatography. Purified antibodies were treated with an excess (10 molar equivalents) of a reducing agent TCEP (tris(2-carboxyethyl)phosphine) at 37° C. for 1-3 hours in a buffered aqueous solution at pH 7.2 containing 2 mM EDTA. The TCEP was removed by passing the reduced variant antibody through a Sephadex G-25 or an ion-exchange column. The reduction of the antibody was confirmed on an analytical reverse phase HPLC system. The purified, reduced antibody was treated with an excess of a disulfide re-oxidising reagent (10 molar equivalents) such as, dhAA (dehydroascorbic acid), CuSO4 (copper(II) sulfate), air, H202 (hydrogen peroxide), N-CS (N-chlorosuccinimide), or O₂ (molecular oxygen) at 4° C. or room temperature for 0.5-24 h in a buffered aqueous solution (pH 7.0). The ratio of free thiols per antibody was estimated by determining the protein concentration from absorption of the protein solution at 280 nm, and the thiol concentration from reaction of the protein with DTDP (dithiodipyridine). The re-oxidation of the antibody was monitored on an analytical reverse phase HPLC and aggregation levels on an analytical size-exclusion column.

After reduction and re-oxidation as described above, the antibody in buffered aqueous solution (pH 7) was treated with 3 molar equivalents of a BM-linker per thiol of antibody containing a cysteine-reactive functional group (maleimide, iodoacetamide, or similar reactive). BM-linkers, typically dissolved in deionized water, was added to the reaction mixture. The reaction was allowed to proceed for 2 hours at room temperature or 4° C. overnight. Afterwards, the conjugate was purified by protein A, ion exchange, size exclusion, or a combination of multiple types of chromatography. Analytical tests such as SDS-PAGE, Western blots, HIC,

Reverse phase HPLC and Mass Spectrometry were carried out to confirm the attachment of the BM linker at the engineered position.

### Example 2—Matriptase Cleavage of Linker Moiety

The following procedure was used for assay for matriptase cleavage of a prodrugged antibody who linker moiety had a matriptase cleavable peptide sequence.

Prodrugged antibody (40 μg) was incubated with 1.3 μg of hMatriptase (30:1, R&D system, 3946-SE-010) in 100 mM Tris buffer, pH7.6 at 37° C. At each time point, 10 μL of sample was mixed with 10 μL quenching buffer (100 mM phosphate buffer with 4M GdnCl and 0.4M TCEP, pH 2.5) to simultaneously deactivate the enzyme and reduce the prodrugged antibody. The quenched sample was subjected to LC/MS analysis.

Example 3—Binding to Activated CD4⁺ T-Cells

Serial dilutions of prodrugged and de-prodrugged (i.e., with linker moiety cleaved) antibody were tested and binding was detected by an APC labelled anti-human IgG secondary antibody. Flow cytometric analyses were performed using a Canto flow cytometer, and the geometric mean fluorescence intensity (GMFI) was determined using FlowJo analysis software. The binding of both test articles was compared with the binding of unprodrugged CTLA4 Ab of SEQ ID NO:1 and NO:2.

### Example 4—IL-2 Secretion Assay

The activity of prodrugged and de-prodrugged antibodies was characterized by an in vitro functional assay using Staphylococcal enterotoxin B (SEB). SEB is a superantigen that strongly activates T cells and stimulates cytokine secretion. Fresh peripheral blood mononuclear cells (PBMC) were isolated from healthy human donor(s) and treated with several concentrations of prodrugged and de-prodrugged antibodies. Simultaneously, a suboptimal concentration (85 ng/mL) of SEB was added to stimulate the cells. T-cell activation was monitoring secretion of the cytokine IL-2 was measured after Day 3 of incubation/treatment.

The following procedure was used: Two buffy coats were collected from healthy donor(s). Whole PBMC were isolated from the buffy coats using a standard Ficoll-Paque separation method of underlaying 15 mL Ficoll for 20 mL buffy coat and spinning for 20 minutes at 2000 rpm with no brake. White interface was separated carefully and washed with PBS several times to remove extra Ficoll and platelets. The cells were then resuspended with T-cell assay media. Serial dilutions of positive control antibody (CTLA4 Ab) from (40 μg/mL to 0.01 μg/mL) and 8 μg/mL to 0.01 μg/ml for the prodrugged and de-prodrugged antibodies were performed and plated in triplicate in a 96-well flat-bottom tissue culture plate. Isolated PBMC were added to the plate at 1×10⁵ cells/well and stimulated by superantigen SEB at 85 ng/mL (a sub-optimal concentration of SEB determined by titrating SEB and by observing the stimulation on T-cell proliferation). The cells were incubated in a 37° C. incubator for 3 days. IL-2 concentration in the supernatants was measured by homogeneous time-resolved fluorescence (HTRF). HTRF data were analyzed using Softmax Pro and graphed using GraphPad Prism software.

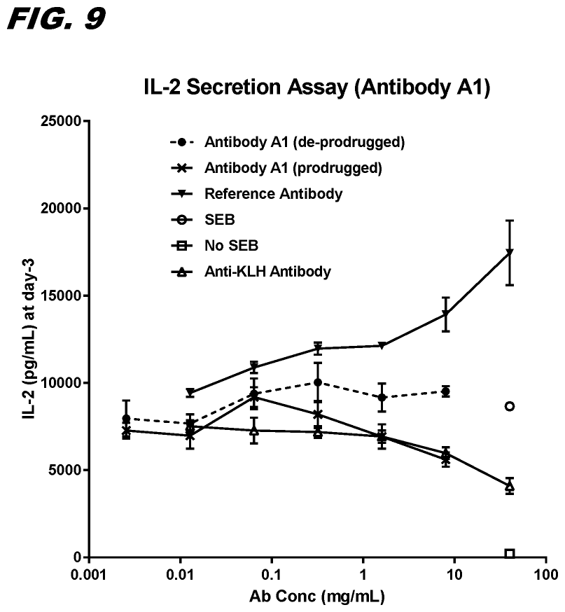

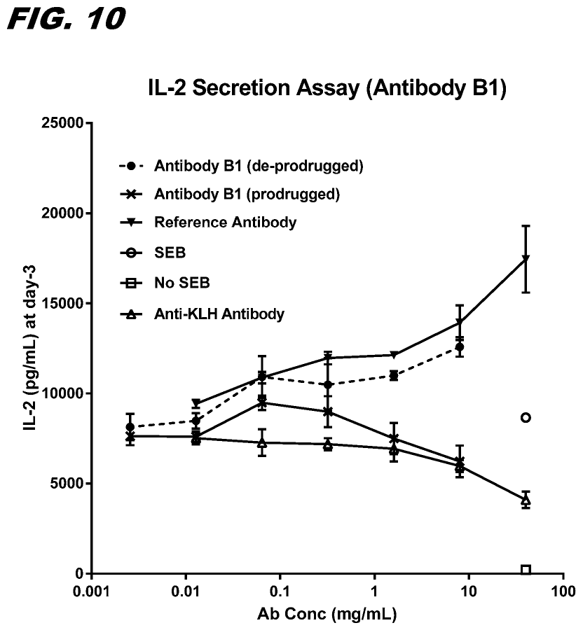

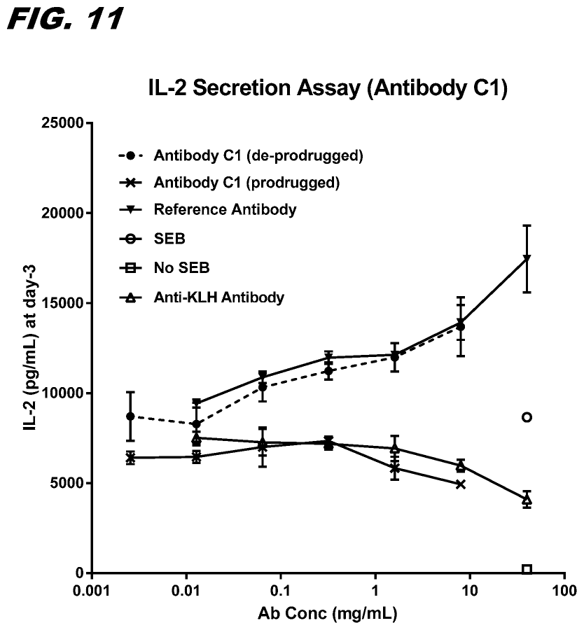

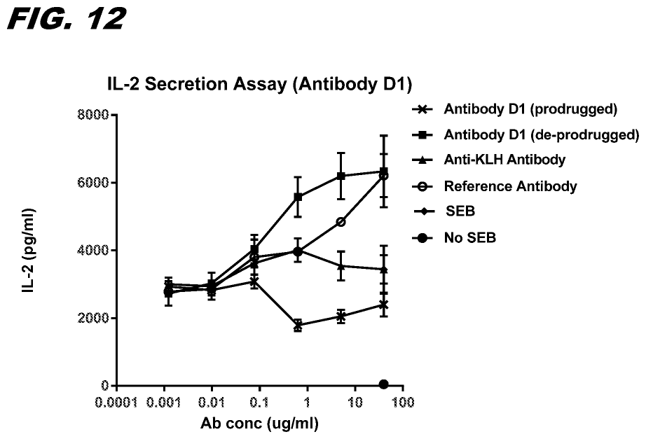

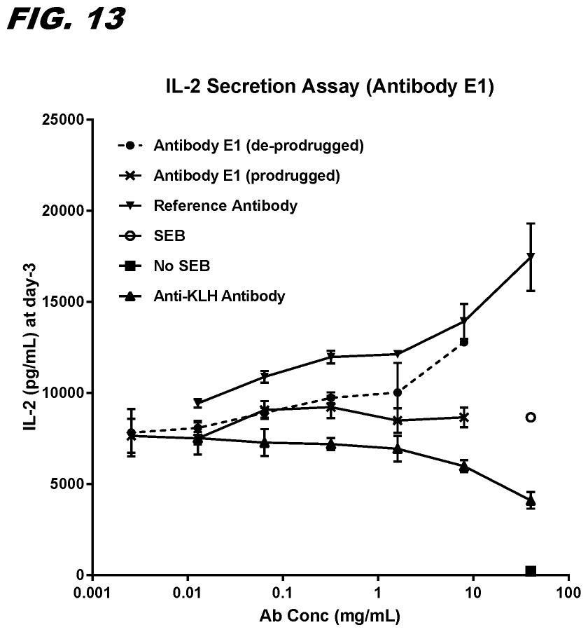

The results are shown in FIGS. 9-13. The control, prodrugged and de-prodrugged anti-CTLA4 antibodies in FIGS. 9-13 were the same as those described in the preceding CD4⁺T cell example.

We observed that the antibodies exhibited dose dependent activity in different donors when compared to the IL-2 secretion elicited by an isotype anti-KLH (keyhole limpet hemocyanin) control antibody. When compared to CTLA4 Ab, the prodrugged antibody showed reduced functional activity. Upon de-prodrugging, there was an approximately 3 fold increase in IL-2 secretion at the highest concentration of the de-prodrugged antibody similar to the postive control of CTLA4 Ab. This result confirms that reducing binding of an antibody by attachment of a blocking moiety reduces activity in a functional T cell assay and that such activity is restored upon removal of the blocking moiety.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments.

Rather, the scope of the invention is defined by the appended claims.

### ACRONYMS AND ABBREVIATIONS

This is a list of acronyms and abbreviations for this specification, along with their meanings.

| ACRONYM OR | |
|---|---|
| ABBREVIATION | MEANING OR DEFINITION |
| Alloc | Allyloxycarbonyl |
| Boc | t-Butyloxycarbonyl |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hünig's base |
| DMA | N,N-Dimethylacetamide |
| DMAP | 4-(Dimethylamino)pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenylmethyloxycarbonyl |
| HATU | (3H-[1,2,3]triazolo[4,5-b]pyridin-3-yl)-1,1,3,3- |
| | tetramethylisouronium hexafluorophosphate (V); |
| | also known as 1-[Bis(dimethylamino)methylene]- |
| | 1H-1,2,3-triazolo[4,5-b]pyridinium 3-oxid |
| | hexafluorophosphate (IUPAC) |
| HEPES | 4-(2-Hydroxyethyl)piperazine-1-ethanesulfonic |
| | acid, N-(2-Hydroxyethyl)piperazine-N′-(2- |
| | ethanesulfonic acid) |
| Hunig's base | See DIPEA, DIEA |
| LCMS | Liquid chromatography mass spectrometry |
| mCPBA | m-chloroperbenzoic acid |
| MsCl | Methanesylfonyl chloride, mesyl chloride |
| PEG | Poly(ethylene glycol) |
| RT | Room temperature, circa 25° C. |
| TBDPS | tert-Butyldiphenylsilyl |
| TBS | t-Butyldimethylsilyl group |
| TEA | Triethylamine |
| TEAA | Triethylammonium acetate |
| TFA | Trifluoroacetic acid |
| THF | Tetrahydrofuran |

### REFERENCES

Full citations for the following references cited in abbreviated fashion by first author (or inventor) and date earlier in this specification are provided below. Each of these references is incorporated herein by reference for all purposes.

Daugherty et al., U.S. Pat. No. 9,169,321 B2 (2015).

Dennis, WO 2016/179003 A1 (2016).

Desnoyers et al., “Tumor-Specific Activation of an EGFR-Targeting Probody Enhances Therapeutic Index,” Sci. Transl. Med. 2013, 5(207), 1.

Lauermann, U.S. Pat. No. 8,809,504 B2 (2014).

Lowman et al., US 2014/0023664 (2014).

Lowman et al., U.S. Pat. No. 9,120,853 B2 (2015). [2015a].

Lowman et al., US 2015/0079088 A1 (2015). [2015b].

Lowman et al., U.S. Pat. No. 9,540,440 B2 (2017).

Moore et al., US 2015/0087810 A1 (2015).

Moore et al., US 2016/0289324 A1 (2016).

Moore et al., U.S. Pat. No. 9,562,073 B2 (2017).

Polu and Lowman, “Probody therapeutics for targeting antibodies to diseased tissue,” Expert Opin. Biol. Ther. 2014, 14(8), 1049.

Rodeck et al., US 2010/0189727 A1 (2010).

Sagert et al., US 2016/0355592 A1 (2016). [2016a].

Sagert et al., US 2016/0355599 A1 (2016). [2016b].

Stagliano et al., U.S. Pat. No. 8,399,219 B2 (2013).

Stagliano et al., U.S. Pat. No. 9,453,078 B2 (2016).

Tipton et al., US 2017/0044259 A1 (2017).

Tomasi et al., U.S. Pat. No. 4,732,863 (1988).

Trouet et al., US 2004/0014652 A1 (2004).

Waldmann et al., US 2013/0028893 A1 (2013).

Waldmann et al., U.S. Pat. No. 8,623,357 B2 (2014).

Wang et al., US 2016/0009817 A1 (2016).

West et al., U.S. Pat. No. 9,127,053 B2 (2015).

West et al., U.S. Pat. No. 9,487,590 B2 (2016). [2016a].

West et al., US 2016/0311903 A1 (2016). [2016b].

West et al., US 2016/0355587 A1 (2016). [2016c].

Williams et al., U.S. Pat. No. 9,193,791 B2 (2015).

TABLE OF SEQUENCES
Table 2 - Sequence Summary

| SEQ ID NO: | SEQUENCE DESCRIPTION |
|---|---|
| 1 | Antibodies A-E V_{H} CDR1 a.a. |
| 2 | Antibodies A-E V_{H} CDR2 a.a. |
| 3 | Antibodies A-E V_{H} CDR3 a.a. |
| 4 | Antibodies A-E V_{L} CDR1 a.a. |
| 5 | Antibodies A-E V_{L} CDR2 a.a. |
| 6 | Antibodies A-E V_{L} CDR3 a.a. |
| 7 | Reference Antibody heavy chain a.a. |
| 8 | Reference Antibody light chain a.a |
| 9 | Antibodies A1-E1 heavy chain a.a. |
| 10 | Antibodies A1-E1 light chain a.a. |

What is claimed is:

1. An anti-CTLA4 antibody having
(i) a heavy chain CDR1 comprising the sequence of amino acids of SEQ ID NO:1,
(ii) a heavy chain CDR2 comprising the sequence of amino acids of SEQ ID NO:2,
(iii) a heavy chain CDR3 comprising the sequence of amino acids of SEQ ID NO:3,
(iv) a light chain CDR1 comprising the sequence of amino acids of SEQ ID NO:4,
(v) a light chain CDR2 comprising the sequence of amino acids of SEQ ID NO:5, and
(iv) a light chain CDR3 comprising the sequence of amino acids of SEQ ID NO:6;
wherein one of Xaa at position 8 of SEQ ID NO:₂, at position 5 of SEQ ID NO:4, at position 8 of SEQ ID NO:4, at position 3 of SEQ ID NO:5, or at position 7 of SEQ ID NO:5 is Cys.

2. An antibody according to claim 1, wherein Xaa at position 8 of SEQ ID NO:2 is Cys.

3. An antibody according to claim 1, wherein Xaa at position 5 of SEQ ID NO:4 is Cys.

4. An antibody according to claim 1, wherein Xaa at position 8 of SEQ ID NO:4 is Cys.

5. An antibody according to claim 1, wherein Xaa at position 3 of SEQ ID NO:5 is Cys.

6. An antibody according to claim 1, wherein Xaa at position 7 of SEQ ID NO:5 is Cys.

7. A prodrugged antibody according to formula (I)
(BM−L)ₘ−Ab (I)
wherein
Ab is an antibody having
(i) a heavy chain CDR1 comprising the sequence of amino acids of SEQ ID NO:1,
(ii) a heavy chain CDR2 comprising the sequence of amino acids of SEQ ID NO:2,
(iii) a heavy chain CDR3 comprising the sequence of amino acids of SEQ ID NO:3,
(iv) a light chain CDR1 comprising the sequence of amino acids of SEQ ID NO:4,
(v) a light chain CDR2 comprising the sequence of amino acids of SEQ ID NO:5, and
(iv) a light chain CDR3 comprising the sequence of amino acids of SEQ ID NO:6;
wherein one of Xaa at position 8 of SEQ ID NO:₂, at position 5 of SEQ ID NO:4, at position 8 of SEQ ID NO:4, at position 3 of SEQ ID NO:5, or at position 7 of SEQ ID NO:5 is Cys;
BM is a blocking moiety that inhibits binding of Ab to its antigen;
each L is, independently, a linker moiety bonded to BM and Ab, L comprising a cleavable moiety and being bonded to Ab at a Cys at position 8 of SEQ ID NO:₂, at position 5 of SEQ ID NO:4, at position 8 of SEQ ID NO:4, at position 3 of SEQ ID NO:5, or at position 7 of SEQ ID NO:5; and
m is 1 or 2.

8. A prodrugged antibody according to claim 7, wherein, in antibody Ab, Xaa at position 8 of SEQ ID NO:2 is Cys.

9. A prodrugged antibody according to claim 7, wherein, in antibody Ab, Xaa at position 5 of SEQ ID NO:4 is Cys.

10. A prodrugged antibody according to claim 7, wherein, in antibody Ab, Xaa at position 8 of SEQ ID NO:4 is Cys.

11. A prodrugged antibody according to claim 7, wherein, in antibody Ab, Xaa at position 3 of SEQ ID NO:5 is Cys.

12. A prodrugged antibody according to claim 7, wherein, in antibody Ab, Xaa at position 8 of SEQ ID NO:5 is Cys.

13. A prodrugged antibody according to claim 7, wherein blocking moiety BM is a poly(ethylene glycol).

14. A prodrugged antibody according to claim 13, wherein the poly(ethylene glycol) has a molecular weight of at least about 2 kDa.

15. A prodrugged antibody according to claim 7, wherein cleavable moiety in L is an enzymatically cleavable peptide.

16. A prodrugged antibody according to claim 15, wherein the enzymatically cleavable peptide is cleavable by at least one enzyme selected from the group consisting of fibroblast activation protein (FAP), urokinase-type plasminogen activator (uPA, urokinase), MT-SP1/matriptase, legumain, and a matrix metalloprotease.

17. A prodrugged antibody according to claim 16, wherein the enzyme is matriptase.

18. A prodrugged antibody according to claim 15, wherein the enzymatically cleavable peptide is LSGRSDNH (SEQ ID NO:5), LSGX (SEQ ID NO:16) or LSGK (SEQ ID NO:16)

19. A prodrugged antibody according to claim 7, having a structure according to formula (IIa) to (IIh): 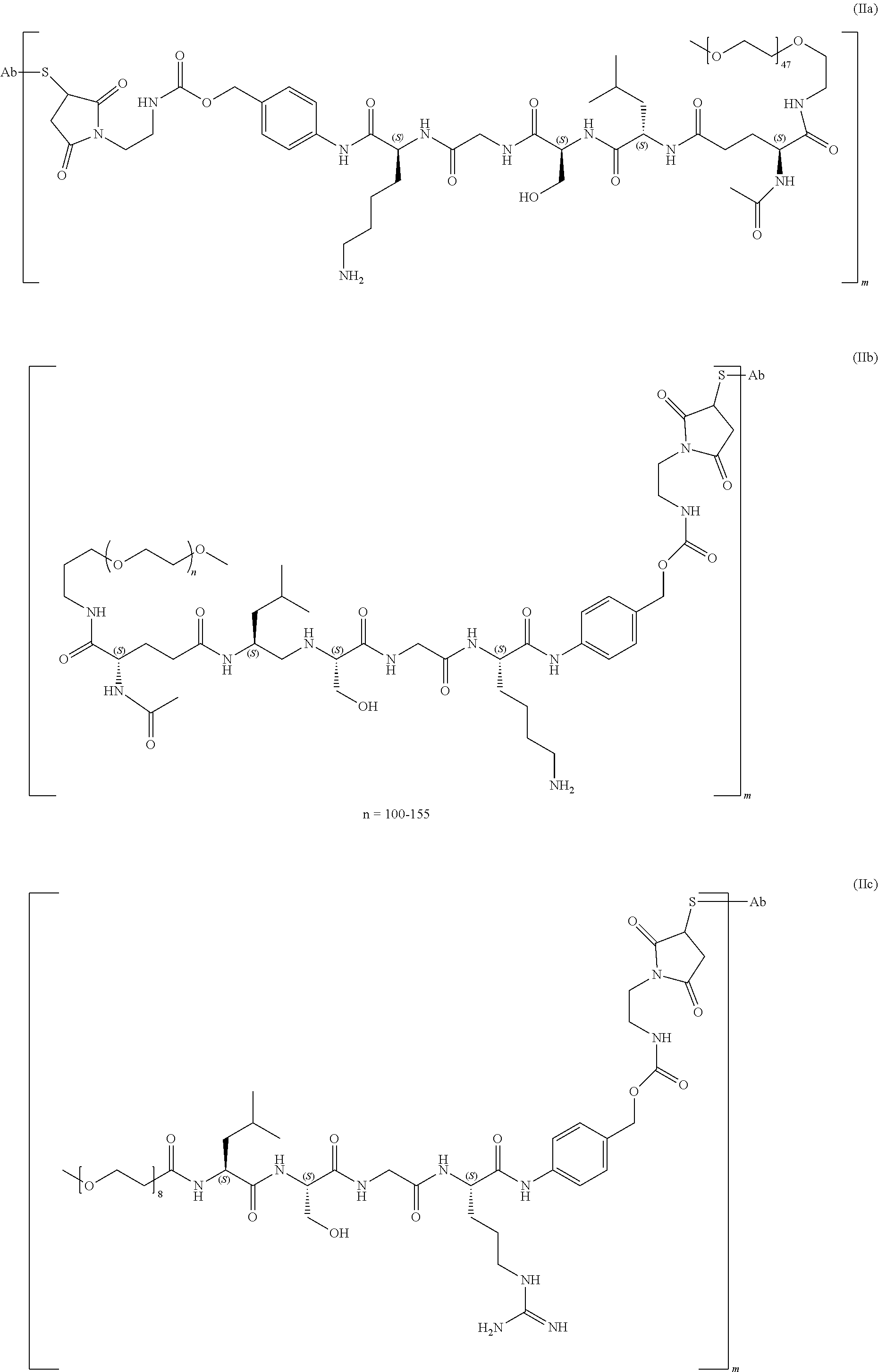 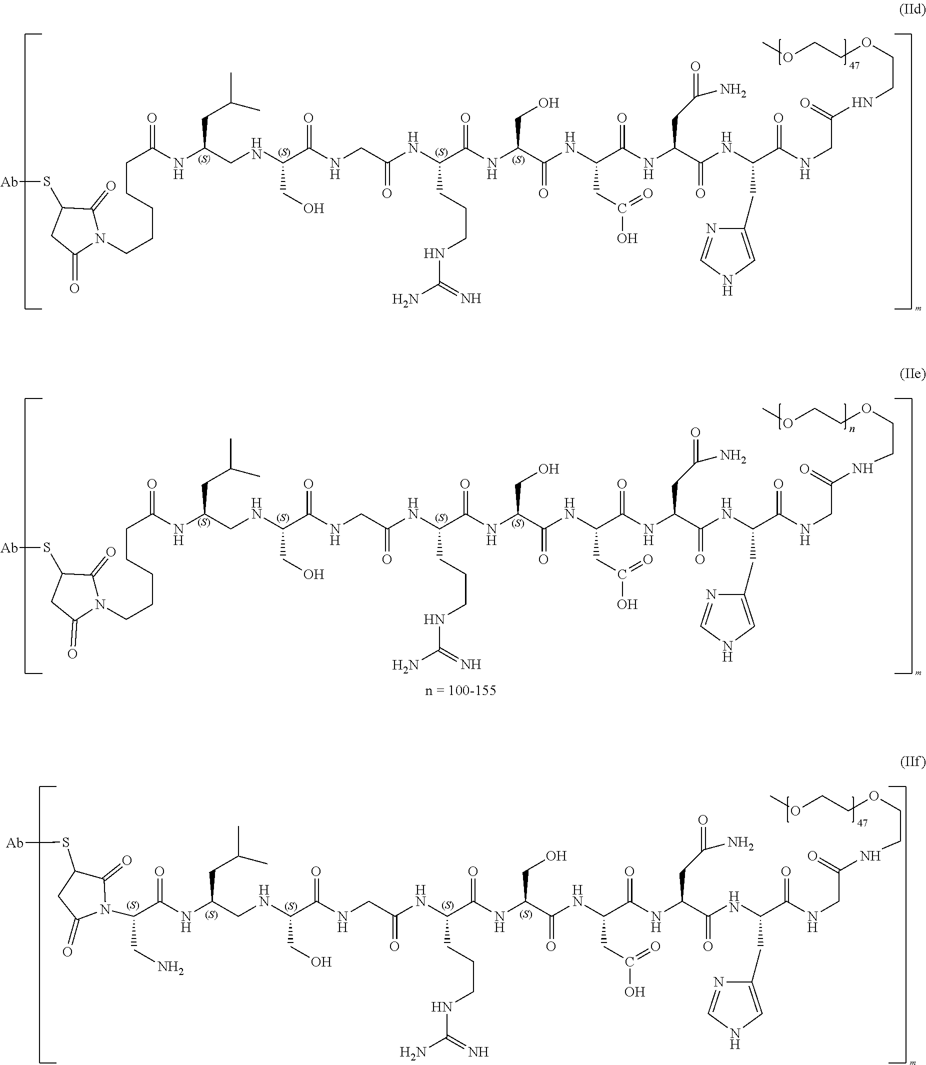 [chemical structure]
